(19) United States
(12) Patent Application Publication
(10) Pub. No.: US 2007/0293893 A1
(43) Pub. Date: Dec. 20, 2007

(54) METHOD AND APPARATUS FOR PRECONDITIONING OF CELLS

(76) Inventors: Craig Stolen, New Brighton, MN (US); Jihong Qu, Maple Grove, MN (US)

Correspondence Address: SCHWEGMAN, LUNDBERG & WOESSNER, P.A., P.O. BOX 2938, MINNEAPOLIS, MN, 55402, US

(21) Appl. No.: 11/424,066

(22) Filed: Jun. 14, 2006

(51) Int. Cl. A61N 1/00

(52) U.S. Cl. 607/2

(57) ABSTRACT

An in vitro cell conditioning system allows for electrical conditioning of cells in preparation for a cell therapy. Electrical stimulation pulses are delivered to the cells placed in a culturing medium. Following the electrical conditioning, the cells are selected according to predetermined criteria. In one embodiment, apoptotic cells are removed from the conditioned cells. In another embodiment, cells with specified surface marker expression are selected. The selected cells are prepared for implantation into living tissue.

### CROSS-REFERENCE TO RELATED APPLICATIONS

This application is related to co-pending, commonly assigned U.S. patent application Ser. No. 10/722,115, “METHOD AND APPARATUS FOR CELL AND ELECTRICAL THERAPY OF LIVING TISSUE,” filed on Nov. 25, 2003, which is hereby incorporated by reference.

### TECHNICAL FIELD

This document relates generally to cell therapy and particularly to a system for in vitro conditioning of cells by electrical stimulation before administering the cells into living tissue in a cell therapy.

### BACKGROUND

The heart is the center of a person's circulatory system. It includes an electromechanical system performing two major pumping functions. The left portions of the heart draw oxygenated blood from the lungs and pump it to the organs of the body, including the heart itself, to provide the organs with their metabolic needs for oxygen. The right portions of the heart draw deoxygenated blood from the body organs and pump it to the lungs where the blood gets oxygenated. These pumping functions are resulted from contractions of the myocardium. In a normal heart, the sinoatrial node generates electrical impulses that propagate through an electrical conduction system to various regions of the heart to excite the myocardial tissues of these regions. Coordinated delays in the propagations of the electrical impulses in a normal electrical conduction system cause the various portions of the heart to contract in synchrony to result in efficient pumping functions. A blocked or otherwise abnormal electrical conduction and/or deteriorated myocardial tissue cause dysynchronous contraction of the heart, resulting in poor hemodynamic performance, including a diminished blood supply to the heart and the rest of the body. The condition where the heart fails to pump enough blood to meet the body's metabolic needs is known as heart failure.

Myocardial infarction (MI) is the necrosis of portions of the myocardial tissue resulted from cardiac ischemia, a condition in which the myocardium is deprived of adequate oxygen and metabolite removal due to an interruption in blood supply caused by an occlusion of a blood vessel such as a coronary artery. The necrotic tissue, known as infarcted tissue, loses the contractile properties of the normal, healthy myocardial tissue. Consequently, the overall contractility of the myocardium is weakened, resulting in an impaired hemodynamic performance. Following an MI, cardiac remodeling starts with expansion of the region of infarcted tissue and progresses to a chronic, global expansion in the size and change in the shape of the entire left ventricle. The consequences include a further impaired hemodynamic performance and a significantly increased risk of developing heart failure, as well as a risk of suffering recurrent MI.

The heart is not capable of repairing the damaged myocardium by itself after an MI. Cardiac muscle cells (cardiomyocytes) cannot naturally regenerate or cannot naturally regenerate in sufficient quantities to replace the dead cardiac muscle cells in the infracted tissue. One way to treat the damaged myocardium is to provide pharmaceutical therapies in an effort to restore heart function. Such therapies may not be particularly effective if the damage to the myocardium is too severe, and pharmaceutical therapy is not believed to regenerate cardiac muscle cells, but instead acts to block or promote certain molecular pathways that are thought to be associated with the progression of heart disease to heart failure. Another treatment for damaged myocardium is called “cell therapy.” Cell therapy involves the administration of endogenous, autologous and/or nonautologous cells to a patient. For example, myogenic cells can be injected into damaged cardiac tissue with the intent of replacing damaged cardiac muscle cells or improving the mechanical properties of the damaged region. However, the administration of myogenic cells does not ensure that the cells will engraft or survive, much less function, and there is a need in the art for enhanced efficacy of cell therapies.

### SUMMARY

An in vitro cell conditioning system allows for preconditioning of cells in preparation for cell therapy. Exemplary stimuli to condition the cells include but are not limited to electrical stimulation, hypoxic or ischemic conditions, oxidative stress or strain. For example, electrical stimulation pulses are delivered to cells placed in a culturing medium. In one embodiment, stem cells are preconditioned with electrical pacing, which regulates cardiac cell specific genes, resulting in cells having pacing ability (cardiac bio-pacers). Following the electrical conditioning, the cells are selected according to predetermined criteria. The selected cells are prepared for implantation into living tissue. In one embodiment, stimuli employed to precondition cells in vivo may be employed at, after, or at and after, implantation of cells, which cells optionally were preconditioned in vitro.

In one embodiment, a system for in vitro conditioning of mammalian cells prior to administration of the mammalian cells into living tissue includes a culturing container; first and second electrodes, and an electrical stimulator. The culturing container contains the mammalian cells and a culturing medium. The first and second electrodes allow for delivering electrical stimulation pulses to the mammalian cells and include two conductive sheets placed substantially parallel to each other. During operation of the system, the two conductive sheets are on substantially horizontal planes. The electrical stimulator delivers the electrical stimulation pulses through the first and second electrodes.

This Summary is an overview of some of the teachings of the present application and not intended to be an exclusive or exhaustive treatment of the present subject matter. Further details about the present subject matter are found in the detailed description and appended claims. Other aspects of the invention will be apparent to persons skilled in the art upon reading and understanding the following detailed description and viewing the drawings that form a part thereof. The scope of the present invention is defined by the appended claims and their legal equivalents.

### BRIEF DESCRIPTION OF THE DRAWINGS

The drawings illustrate generally, by way of example, various embodiments discussed in the present document. The drawings are for illustrative purposes only and may not be to scale.

FIG. 1 is an illustration of an embodiment of an in vitro cell conditioning system.

FIG. 2 is an illustration of another embodiment of the in vitro cell conditioning system.

FIG. 3 is an illustration of another embodiment of the in vitro cell conditioning system.

FIG. 4 is a block diagram illustrating an embodiment of an electrical stimulator of the in vitro cell conditioning system.

FIG. 5 is a block diagram illustrating a specific embodiment of the electrical stimulator.

FIG. 6 is a block diagram illustrating an embodiment of another in vitro cell conditioning system.

FIG. 7 is a flow chart illustrating an embodiment of a method for in vitro electrical conditioning of cells prior to administration of the cells into living tissue.

FIG. 8 is a flow chart illustrating a specific embodiment of the method of FIG. 7.

FIG. 9 is a flow chart illustrating another specific embodiment of the method of FIG. 7.

FIG. 10 is a flow chart illustrating another specific embodiment of the method of FIG. 7.

### DETAILED DESCRIPTION

In the following detailed description, reference is made to the accompanying drawings which form a part hereof, and in which is shown by way of illustration specific embodiments in which the invention may be practiced. These embodiments are described in sufficient detail to enable those skilled in the art to practice the invention, and it is to be understood that the embodiments may be combined, or that other embodiments may be utilized and that structural, logical and electrical changes may be made without departing from the spirit and scope of the present invention. References to “an”, “one”, or “various” embodiments in this disclosure are not necessarily to the same embodiment, and such references contemplate more than one embodiment. The following detailed description provides examples, and the scope of the present invention is defined by the appended claims and their legal equivalents.

This document discusses in vitro preconditioning of cells before the implantation of the cells into living tissue for cell therapy. The cell therapy includes implantation of cells into living tissue to prevent or inhibit disease progression and optionally replace or augment diseased or injured tissue or cells. For example, cells are administered into the heart to prevent progression of, as well as repairing tissue damage resulting from, cardiac disorders including MI, heart failure, aberrant pacing or conduction, or arrhythmias. In another embodiment, cells are administered to the spinal cord or a vagal nerve to prevent or inhibit progression of neural degeneration or enhance neural connections. The efficacy of cell therapy is associated with, for example, retention, engraftment, survival, differentiation, integration, and/or function of the implanted cells in the recipient. The preconditioning includes but is not limited to electrical stimulation, hypoxic conditions, ischemic conditions, oxidative stress or strain conditioning of the cells in a culturing medium, for instance, to enhance one or more desirable properties of the cells. After the preconditioning, a subpopulation of cells may be selected from the preconditioned cells based on predetermined criteria prior to implantation into the living tissue. The selected cells have altered properties relative to the unselected subpopulation of preconditioned cells that enhance, for example, the retention, engraftment, survival, differentiation, integration, and/or function of the cells after being implanted into the living tissue.

The in vitro electrical conditioning includes subjecting the cells to electrical conditions that simulate the electrical conditions in the myocardium or other systems, such as neurological systems. In the heart, contraction results primarily from the contractions of atrial and ventricular muscle fibers. Contraction of atrial and ventricular muscle fibers is slower and is of a longer duration than the contraction of skeletal muscle. Cardiac muscle and skeletal muscle, however, share a number of common anatomic characteristics. In the same manner as skeletal muscle, cardiac muscle is made up of elongated fibers with transverse dark and light bands. The dark bands correspond to the boundaries between cells. Each fiber is made up of individual cells connected in series with each other. Cardiac muscle includes myofibrils, which are the longitudinal parallel contractile elements composed of actin and myosin filaments that are almost identical to those of the skeletal muscle. The actin and myosin filaments interdigitate and slide along each other during contraction. Contraction is caused by action potentials that propagate along or spread over the muscle fibers. The propagation of action potentials results from changes in the electrical potential across muscle cell membranes, referred to as membrane potential. The changes in the membrane potential are in turn caused by flow of sodium, potassium, and/or calcium ions across the muscle cell membranes through ion channels, which are formed by protein molecules in the cell membranes. Some types of muscle include protein structures called gap junctions through which ions flow from one muscle cell to another. Gap junctions allow the flow of ions, and hence the propagation of action potentials, directly from one cell to another. Cardiac muscle has at least two unique anatomic characteristics: a high density of calcium-sodium channels and a high density of gap junctions. These characteristics distinguish cardiac muscle from skeletal and other types of muscle.

Action potential propagates in skeletal muscle mainly via the sudden opening of fast sodium channels that allow sodium ions to enter the muscle cells. Each opening of a fast sodium channel lasts for only a few ten-thousandths of a second. In contrast, cardiac muscle includes both fast sodium channels and slow calcium-sodium channels that allow both calcium and sodium to enter the muscle cells. Each opening of a slow calcium-sodium channel lasts for several tenths of a second. This results in the long duration of contraction, which characterizes cardiac muscle.

Gap junctions in cardiac muscle fibers allow relatively free flow of ions across the cell membranes along the fiber axes. Thus, action potentials travel from one cell to another with little resistance. Cardiac muscle is a syncytium (mass of fused cells) with muscle fibers arranged in a latticework in which the fibers branch, merge, and branch again. When one cell in the syncytium becomes excited, the action potential propagates from cell to cell and spreads throughout the latticework interconnections. The heart includes two syncytiums, the atrial syncytium and the ventricular syncytium. In a normal heart, action potentials are conducted from the atrial syncytium to the ventricular syncytium through a conduction system, the A-V bundle, and the atrial syncytium contracts before the ventricular syncytium.

In one embodiment, the electrical preconditioning includes delivering electrical stimulation pulses such as cardiac pacing pulses to the cells in culture so as to induce those cells to differentiate into cardiac progenitor cells, e.g., pre-contractile cells. In one embodiment, electrical preconditioning may result in the cells proliferating and differentiating into cardiac muscle cells, and preferably results in cells functioning as cardiac muscle cells.

In one embodiment, the in vitro electrical conditioning of the cells results in cells with one or more characteristics of cardiac muscle cells, including a high density of calcium-sodium channels, a high density of gap junctions or a combination thereof. Moreover, once those cells are implanted in the myocardium, they are subject to the pattern of contractions in the myocardium and may, if they are not cardiac muscle cells, differentiate into cardiac muscle cells. Electrical conditioning of the cells, including cardiac pacing, may result in an altered expression profile of the cells, including increased calcium-sodium channel expression and/or increased expression and/or formation of gap junctions. For instance, electrical conditioning may increase angiotensin II or VEGF expression, which in turn increases gap junction formation.

In one embodiment, the in vitro electrical conditioning of the cells results in cells with one or more characteristics of cardiac progenitor cells, including but not limited to expression of genes associated with cardiac cell differentiation, e.g., homeobox genes or factors such as dkk-1, hex, hop, GATA-4, MEF2, and eHAND, or other characteristics of cells induced to commit to differentiating into cardiac cells (cardioinduction).

In another embodiment, the electrical preconditioning includes delivering electrical stimulation pulses to the cells in culture so as to induce those cells to differentiate into neural progenitor cells (neuroinduction). In one embodiment, electrical conditioning may result in the cells proliferating and optionally differentiating into neural cells, for instance, neurons, glial cells, astrocytes, or oligodendrocytes, or pancreatic beta cells.

In one embodiment, the in vitro electrical conditioning of the cells results in cells with one or more characteristics of neural cells. Neural cells or other electrically responsive cells such as pancreatic beta cells may be useful to inhibit or treat diseases including but not limited to spinal cord injury, brain injury, and neurodegenerative diseases such as Alzheimer's disease, as well as diabetes. Moreover, once the cells are implanted into a mammal, e.g., into the spinal cord, brain or pancreas, they are subject to endogenous stimuli which may result in formation of neural networks with existing neural cells, differentiation into more differentiated cells, e.g., neurons, glial cells, astrocytes, or oligodendrocytes, pancreatic beta cells, or a combination thereof. Electrical conditioning of the cells, including pacing, may result in an altered expression profile of the cells. For instance, electrical conditioning may increase gene products associated with the nervous system such as CD133, glial fibrillary acidic protein, microtubule associated protein-2, myelin basic protein, nestin, neural tubulin, neurofilament, neurosphere, noggin, O4, O1, synaptophysin, and tau, or gene products associated with the pancreas, such as cytokeratin 19, glucagon, insulin, insulin promoting factor-1, nestin, pancreatic polypeptide or somatostatin.

In one embodiment, electrical preconditioning includes delivering electrical stimulation pulses to the cells in culture so as to result in transdifferentiation of the cells, e.g., from one type of progenitor cell to another.

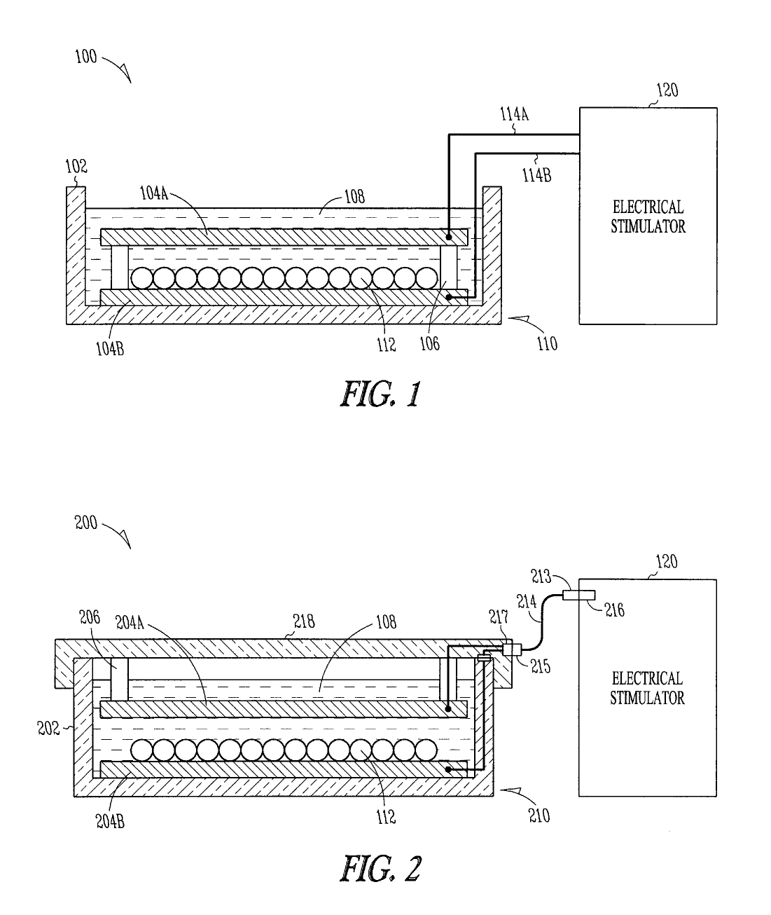

FIG. 1 is an illustration of an embodiment of an in vitro cell conditioning system 100. System 100 is used to prepare cells prior to their administration into living tissue. System 100 includes a culturing module 110 and an electrical stimulator 120. Culturing module 110 includes a culturing container 102 and electrodes 104A-B. Culturing container 102 is used to host cells 112 and a physiologic culturing medium 108. Examples of cells 112 include autologous or nonautologous cells, e.g., allogeneic or xenogeneic cells, and including adult stem cells such as bone marrow cells, endothelial progenitor cells, cardiac progenitor cells, and adipose derived stem cells, as well as neural stem cells, skeletal myoblasts, embryonic stem cells, cloned cells such as those prepared by somatic cell nuclear transfer, and cells obtained from cord or menstrual blood. Examples of sources and isolation of such cells are discussed in U.S. patent application Ser. No. 10/722,115, “METHOD AND APPARATUS FOR CELL AND ELECTRICAL THERAPY OF LIVING TISSUE,” filed on Nov. 25, 2003, assigned to Cardiac Pacemakers, Incorporated, which is incorporated herein by reference in its entirety. Cells subjected to electrical conditioning may be unmodified cells (not genetically altered) or genetically altered cells (e.g., transgenic or a gene “knock out”). The cells may be genetically altered before, during, or after electrical conditioning. Electrodes 104A-B are placed in culturing container 102 and electrically connected to electrical stimulator 120 through conductors 114A-B.

Electrodes 104A-B allow for delivery of electrical stimulation to cells 112. In one embodiment, the delivery of electrical stimulation includes delivery of electrical stimulation pulses. In one embodiment, cells 112 are placed on a surface, and electrodes 104A-B are arranged to allow substantially uniform distribution of electrical energy over the surface when the electrical stimulation pulses are delivered. In one embodiment, when system 100 is in use, the surface is on a substantially horizontal plane. In one embodiment, electrodes 104A-B include two plate electrodes placed substantially parallel to each other and on substantially horizontal planes. The two plate electrodes include two conductive sheets. In various specific embodiments, the two conductive sheets are each made of a metallic material, such as stainless steel, titanium, and platinum-iridium. In a specific embodiment, cells 112 are placed on a surface of one of the conductive sheets. The two conductive sheets are separated by a distance of approximately 0.1 to 10 millimeters. In a specific embodiment, the two conductive sheets are configured to fit into a standard tissue/cell culturing dish. In a specific embodiment, the two conductive sheets are separated by non-conductive spacers 106. In various embodiments, the two conductive sheets are completely immersed in culturing medium 108. To apply the electrical stimulation to cells 112, electrodes 104A is electrically connected to electrical stimulator 120 through conductor 114A, and electrodes 104B is electrically connected to electrical stimulator 120 through conductor 114B. Electrical stimulator 120 delivers the electrical stimulation pulses to cells 112 through electrodes 104A-B.

FIG. 2 is an illustration of an embodiment of an in vitro cell conditioning system 200. System 200 is used to prepare cells prior to their administration into living tissue and is an alternative embodiment of system 100. System 200 includes a culturing module 210 and electrical stimulator 120. Culturing module 210 includes a culturing container 202 with a lid 218 and electrodes 204A-B. Culturing container 202 is used to host cells 112 and physiologic culturing medium 108. Electrodes 204A-B are placed in culturing container 202 and electrically connected to electrical stimulator 120 through a cable 214.

Electrodes 204A-B allow for delivery of electrical stimulation to cells 112. In one embodiment, the delivery of electrical stimulation includes delivery of electrical stimulation pulses. In one embodiment, cells 112 are placed on a surface, and electrodes 204A-B are arranged to allow substantially uniform distribution of electrical energy over the surface when the electrical stimulation pulses are delivered. In one embodiment, when system 200 is in use, the surface is on a substantially horizontal plane. In one embodiment, electrodes 204A-B include two plate electrodes placed substantially parallel to each other and on substantially horizontal planes. The two plate electrodes include two conductive sheets. In various specific embodiments, the two conductive sheets are each made of a metallic material, such as stainless steel, titanium, and platinum-iridium. In a specific embodiment, cells 112 are placed on a surface of one of the conductive sheets. The two conductive sheets are separated by a distance of approximately 0.1 to 10 millimeters. In a specific embodiment, the two conductive sheets are configured to fit into a standard tissue/cell culturing dish. In the illustrated embodiment, electrode 204B is attached to the bottom of culturing container 202, and electrode 204A is affixed to lid 218, which is configured to cover culturing container 202. In a specific embodiment, electrode 204A is affixed to lid 218 through spacers 206. In various embodiments, the two conductive sheets are completely immersed in culturing medium 108. To apply the electrical stimulation to cells 112, electrodes 104A-B are each electrically connected to electrical stimulator 120 through a conductor of cable 214. Electrical stimulator 120 delivers the electrical stimulation pulses to cells 112 through electrodes 204A-B.

Cable 214 is coupled between connectors 213 and 215. Connector 213 is to be plugged into a connector 216 on electrical stimulator 120. In the illustrated embodiment, connector 214 is to be plugged into a connector 217 on lid 218. Culturing container 202 and lid 218 each include a conductive contact or connector to allow for electrical connection through which the electrical stimulation pulses are delivered to electrode 204B. In an alternative embodiment, connector 215 is to be plugged into a connector on culturing container 202, and culturing container 202 and lid 218 each include a conductive contact or connector to allow for electrical connection through which the electrical stimulation pulses are delivered to electrode 204A.

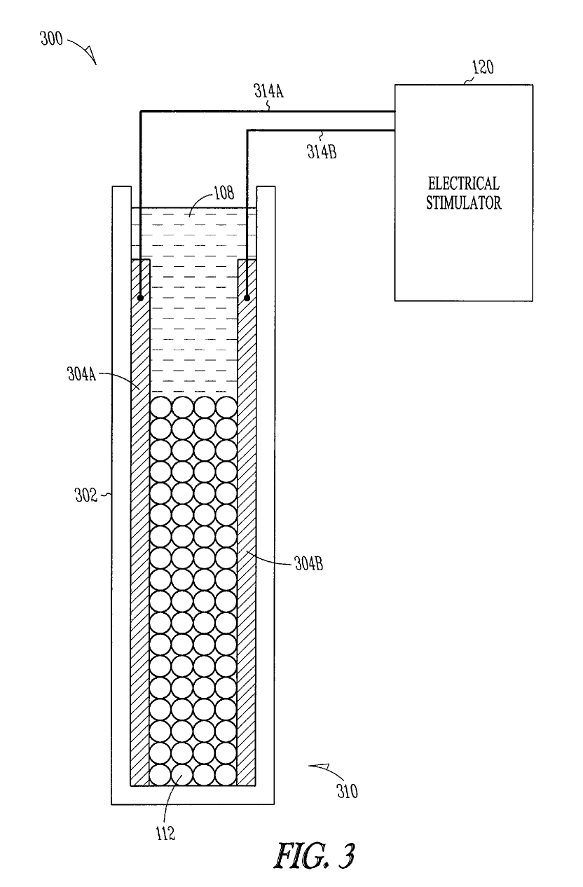

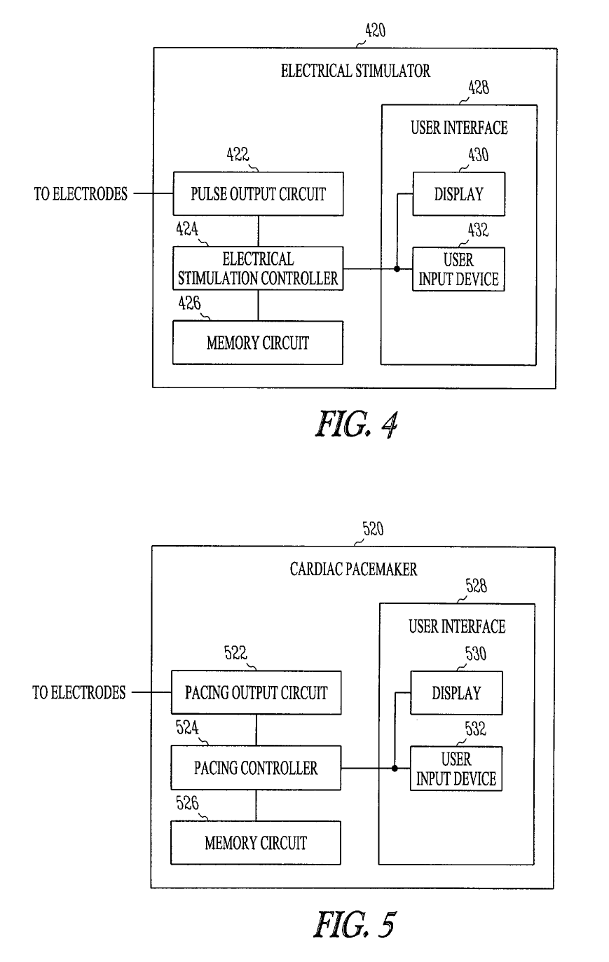

FIG. 3 is an illustration of an embodiment of an in vitro cell conditioning system 300. System 300 is used to prepare cells prior to their administration into living tissue and is another alternative embodiment of system 100. System 300 includes a culturing module 310 and electrical stimulator 120. Culturing module 310 includes a culturing container 302 and electrodes 304A-B. Culturing container 302 is used to host cells 112 and physiologic culturing medium 108. Electrodes 304A-B are placed in culturing container 302 and electrically connected to electrical stimulator 120 through conductors 314A-B.

Electrodes 304A-B allow for delivery of electrical stimulation to cells 112. In one embodiment, the delivery of electrical stimulation includes delivery of electrical stimulation pulses. In one embodiment, when system 300 is in use, electrodes 304A-B include two plate electrodes placed substantially parallel to each other and on substantially vertical planes. The two plate electrodes include two conductive sheets. In various specific embodiments, the two conductive sheets are each made of a metallic material, such as stainless steel, titanium, and platinum-iridium. The two conductive sheets are separated by a distance of approximately 0.1 to 10 millimeters. In a specific embodiment, the two conductive sheets are configured to fit into a standard tissue/cell culturing container. In one illustrated embodiment, electrodes 304A-B are both affixed onto the interior wall of culturing container 302. To apply the electrical stimulation to cells 112, electrodes 304A is electrically connected to electrical stimulator 120 through conductor 314A, and electrodes 304B is electrically connected to electrical stimulator 120 through conductor 314B. Electrical stimulator 120 delivers the electrical stimulation pulses to cells 112 through electrodes 304A-B.

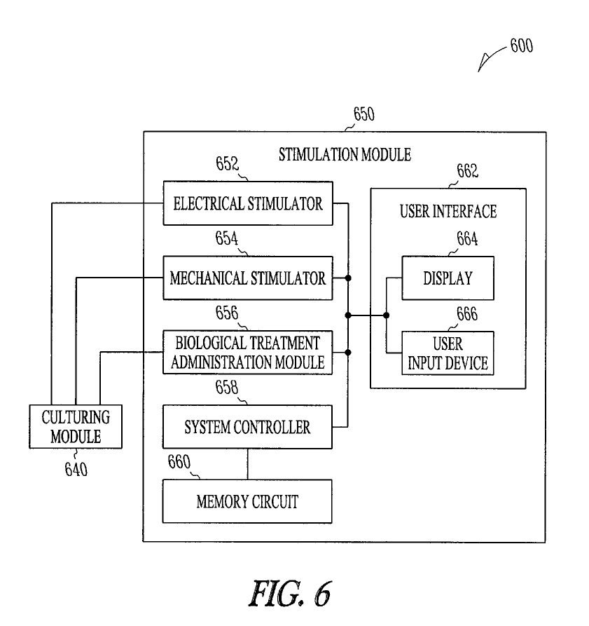

FIG. 4 is a block diagram illustrating an embodiment of an electrical stimulator 420. Electrical stimulator 420 is a specific embodiment of electrical stimulator 120 and includes a pulse output circuit 422, an electrical stimulation controller 424, a memory circuit 426, and a user interface 428.

Pulse output circuit 422 delivers the electrical stimulation pulses to cells 112 though the electrodes (104A-B, 204A-B, or 304A-B). Electrical stimulation controller 424 controls the delivery of the electrical stimulation pulses using a plurality of electrical stimulation parameters. Memory circuit 426 stores one or more values for each of the electrical stimulation parameters. In one embodiment, the electrical stimulation parameters include pulse amplitude, pulse width, stimulation frequency or inter-pulse interval, stimulation duration, and stimulation duty cycle. In various embodiments, the values of the electrical stimulation parameters are determined based on one or more of the objectives of the in vitro conditioning, the type of the cells, and the target organ into which the cells are to be implanted. In various embodiments, the values of the electrical stimulation parameters are programmed to alter gene expression in the cells, e.g., to upregulate cardiac cell specific, neural cell specific, or pancreatic beta cell specific, gene expression, to enhance survival and/or function of the cells after being implanted into living tissue, and/or to enhance one or more of proliferation, differentiation, alignment, conductivity, contractility, and calcium (Ca²⁺) handling of the cells. In one embodiment, the electrical stimulation creates an environment simulating electrical conditions of the living tissue to which cells 112 are subjected to after being implanted. In a specific embodiment, the values of the electrical stimulation parameters are programmed to subject the cells to simulated normal cardiac electrical conditions. In another specific embodiment, the values of the electrical stimulation parameters are programmed to subject the cells to simulated pathological cardiac electrical conditions. Examples of the simulated pathological cardiac electrical conditions include simulated cardiac electrical conditions after myocardial infarction and simulated cardiac electrical conditions associated with heart failure. In a specific embodiment, the values of the electrical stimulation parameters are programmed to subject the cells to simulated normal neurological electrical conditions. In another specific embodiment, the values of the electrical stimulation parameters are programmed to subject the cells to simulated pathological neurological electrical conditions. In a specific embodiment, the values of the electrical stimulation parameters are programmed to subject the cells to simulated normal pancreatic beta cell electrical conditions. In another specific embodiment, the values of the electrical stimulation parameters are programmed to subject the cells to simulated pathological pancreatic beta cell electrical conditions.

User interface 428 allows for control of the in vitro cell conditioning including the programming of the electrical stimulation parameters. User interface 428 includes a display 430 and a user input device 432. Display 430 presents the electrical stimulation parameters with their values. User input device 432 receives the values of the electrical stimulation parameters programmer by a user. In one embodiment, user input device 432 allows the user to program one or more of the pulse amplitude, pulse width, stimulation frequency or inter-pulse interval, stimulation duration, and stimulation duty cycle. In one embodiment, one or more of the electrical stimulation parameters are pre-programmed to fixed values and therefore not user-programmable. In one embodiment, the values of the electrical stimulation parameters are predetermined based on factors such as the objective of the in vitro conditioning, the type of the cells, and the target organ into which the cells are to be implanted. The user selects one or more of such factors using the user input device. Electrical stimulation controller 424 selects the values of the electrical stimulation parameters pre-stored in memory circuit 426 according to the selection.

FIG. 5 is a block diagram illustrating an embodiment of a cardiac pacemaker 520, which is a specific embodiment of electrical stimulator 420. Cardiac pacemaker 520 includes a pacing output circuit 522, a pacing controller 524, a memory circuit 526, and a user interface 528.

Pacing output circuit 522 is a specific embodiment of pulse output circuit 422 and delivers cardiac pacing pulses to cell 112 through the electrodes (104A-B, 204A-B, or 304A-B). Pacing controller 524 is a specific embodiment of electrical stimulation controller 424 and controls the delivery of the cardiac pacing pulses using a plurality of pacing parameters. Memory circuit 526 is a specific embodiment of memory circuit 426 and stores one or more values for each of the pacing parameters. The pacing parameters include pulse amplitude, pulse width, pacing rate, pacing duration, and pacing duty cycle. In various embodiments, each of these pacing parameters has a pre-programmed fixed value or is field-programmable by the user.

User interface 528 is a specific embodiment of user interface 428 and includes a display 530 and a user input device 532. Display 530 is a specific embodiment of display 430 and presents the pacing parameters with their values. User input device 532 is a specific embodiment of user input device 432 and receives the values of the pacing parameters programmer by a user. In one embodiment, user input device 532 allow the user to program one or more of the pulse amplitude, pulse width, pacing rate, pacing duration, and pacing duty cycle. In one embodiment, the pulse amplitude is programmable in a range of approximately 1 to 10 volts/millimeter, the pulse width is programmable in a range of approximately 0.5 to 10 milliseconds, and the pacing rate is programmable in a range of approximately 30 to 600 pulses per minute. The pacing duration ranges from several milliseconds to several hours.

In one embodiment, one or more of the pacing parameters are pre-programmed to fixed values and therefore not user-programmable. In one embodiment, the values of the pacing parameters are predetermined based on factors such as the objective of the in vitro conditioning, the type of the cells, and the target organ into which the cells are to be implanted. The user selects one or more of such factors using the user input device. Pacing controller 524 selects the values of the pacing parameters pre-stored in memory circuit 526 according to the selection.

FIG. 6 is a block diagram illustrating an embodiment of an in vitro cell conditioning system 600. In addition to the electrical conditioning, system 600 allows for treatment of cells including mechanical and/or biological conditioning prior to the administration of the cells into living tissue. Biological conditioning includes subjecting cells to exogenous agents, e.g., differentiation factors, growth factors, angiogenic factors and cytokines, as well as expression cassettes encoding a desirable gene product (transgenes) or cassettes for homologous recombination, e.g., to produce gene knock outs. System 600 includes a culturing module 640 and a stimulation module 650. Culturing module 640 includes a culturing container to contain the cells to be treated and a culturing medium, and structures allowing delivery of electrical stimulation, delivery mechanical stimulation, administration of biological treatment, or a combination thereof. In one embodiment, culturing module 640 includes culturing module 110 and structures allowing delivery mechanical stimulation and administration of biological treatment. Stimulation module 650 includes an electrical stimulator 652, a mechanical stimulator 654, a biological treatment administration module 656, a system controller 658, a memory circuit 660, and a user interface 662.

Electrical stimulator 652 provides for in vitro electrical conditioning of the treated cells, as described in this document. In one embodiment, electrical stimulator 652 includes electrical stimulator 120, including its specific embodiments, electrical stimulator 420 or 520, as discussed above.

Mechanical simulator 654 provides for the in vitro mechanical conditioning of the treated cells. In one embodiment, mechanical stimulator 654 includes a myocardial stress simulator to create a mechanical stress upon the treated cells. The mechanical stress simulates the tension applied upon cardiac muscle cells in the myocardium. The tension results from mechanical forces created by the cyclical changes in heart volume and intracardiac blood pressure. In one embodiment, the treated cells are placed in a culturing substrate including an electrode for the electrical stimulation (such as electrode 104B or 204B) or a separate structure placed between the electrodes for the electrical stimulation (such as electrodes 104A-B or 204A-B). The myocardial stress simulator includes a variable speed motor and a mechanical linkage that provides for the interface between the motor and the culturing substrate, allowing for a controlled motion of the motor to create a calibrated cyclic mechanical tension on the culturing substrate. In one embodiment, the mechanical linkage allows the culturing substrate to be cyclically stretched and relaxed in two or more directions without vibration and hesitation. One example of such a mechanical stimulator is given in Terracio et al., In vitro Cellular & Developmental Biology, 24(1), 53-58, 1988.

Biological treatment administration module 656 provides for in vitro biological conditioning of the treated cells. Biological treatment administration module 656 introduces one or more exogenous agents to the culturing medium, which alter one or more properties of the cells. In one embodiment, biological treatment administration module 656 includes one or more chemical dispensers to allow controlled release of one or more chemical or biochemical agents, e.g., recombinant virus or a liposome containing a plasmid encoding a desirable gene product, the plasmid or other gene delivery vehicle, into the culturing medium. In one embodiment, biological treatment administration module 656 includes an array of dispensers each controlled for releasing a predetermined amount of chemical or biochemical agent(s) into the culturing medium at one or more predetermined times.

System controller 658 controls delivery of the electrical stimulation, delivery of the mechanical stimulation, and administration of the biological treatment using a plurality of parameters stored in a memory circuit 660. In one embodiment, system controller 658 coordinates the operation of the electrical stimulator with one or both of mechanical stimulator 654 and biological treatment administration module 656. In various embodiments, system controller 658 controls the magnitude (or intensity) of each of the electrical, mechanical, and biological stimuli and coordinates the timing for delivering those stimuli. The magnitude of the electrical stimulation is controlled by parameters such as pulse amplitude, pulse width, stimulation frequency or inter-pulse interval, stimulation duration, and stimulation duty cycle. The magnitude of the mechanical stimulation is controlled by parameters such as frequency and degree of cell deformation (such as measured by the extension of cell length by a predetermined percentage of the original cell length). The magnitude of the biological stimulation is controlled by parameters such as volume and concentration of each chemical and/or biochemical agent. The timing is controlled by parameters such as starting times and durations that control the deliveries of all the electrical, mechanical, and/or biological stimuli in a predetermined sequence of stimulation, i.e., the complete in vitro conditioning.

User interface 662 includes a display 664 and a user input device 666. Display 664 informs the user of the status and the progress of the in vitro conditioning, including the parameters being applied. User input device 666 receives commands from the user to control the in vitro conditioning of the treated cells and allows the use to program the values of user-programmable parameters. In one embodiment, culturing module 640 includes a structure, such as a microscope, that allows for monitoring of the treated cells. In a further embodiment, display 664 presents cell reactions observed through the microscope. In other embodiments, a sample of the culture is removed for analysis, e.g., to detect RNA or protein expression patterns, or other detectable phenotypes, e.g., cell size or surface marker expression.

In one embodiment, the user controls the complete in vitro conditioning process, or portions of the process, by entering parameters defining the magnitude and timing of each stimulus and giving a command to deliver one stimulus or one sequence of stimuli through user interface 662. In another embodiment, an instruction set defining a predetermined sequence of electrical, mechanical, and/or biological stimuli, including the required magnitude and timing, is stored in memory circuit 660. System controller 658 controls electrical stimulator 652, mechanical simulator 654, and biological treatment administration module 656 by automatically executing the instruction set. In a further embodiment, system controller 658 allows the user to adjust parameters in the instruction set during the in vitro conditioning in response to cell reactions observed through the microscope or presented on display 664, and/or in response to information gathered via analyses as described above.

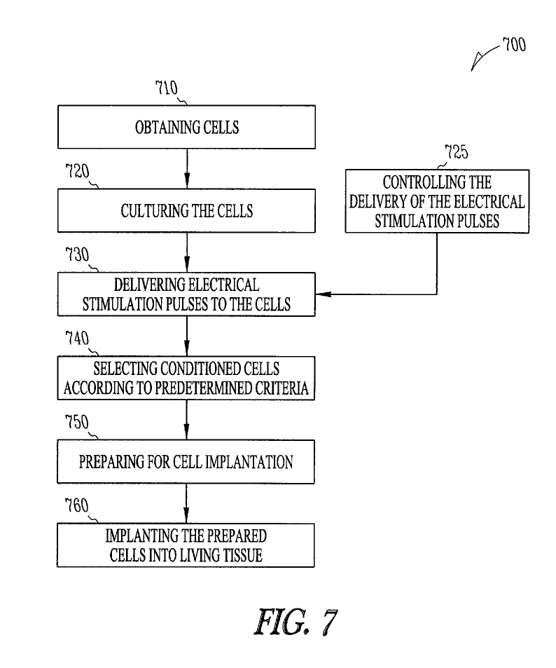

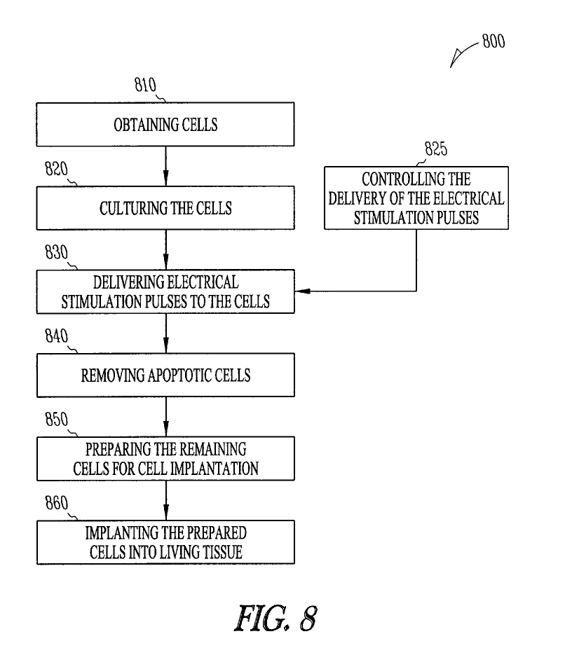

FIG. 7 is a flow chart illustrating an embodiment of a method 700 for in vitro electrical conditioning of cells prior to administration of the cells into living tissue. In one embodiment, in vitro cell conditioning system 100, 200, 300, or 600 is used in the performance of method 700, including steps 720, 725, and 730.

Cells are obtained at 710 for administration into living tissue. Examples of the obtained cells include adult stem cells such as bone marrow cells, endothelial progenitor cells, cardiac progenitor cells, and adipose derived stem cells, as well as neural stem cells, skeletal myoblasts, embryonic stem cells, and cloned cells, e.g., those obtained using somatic cell nuclear transfer, including but not limited to cells discussed in U.S. patent application Ser. No. 10/722,115. In one embodiment, the cells are genetically modified, for instance, by introducing and expressing an open reading frame in an expression vector or introducing a vector to knock out expression of a gene by homologous recombination. The cells are cultured in a physiologic culturing medium at 720. Electrical stimulation pulses are delivered to the cells in the physiologic culturing medium at 730. The delivery of the electrical stimulation pulses are controlled using a plurality of electrical stimulation parameters at 725. Such stimulation parameters include, but are not limited to, one or more of pulse amplitude, pulse width, stimulation frequency, stimulation duration, and stimulation duty cycle. In one embodiment, cardiac pacing pulses are delivered at 730. The delivery of the cardiac pacing pulses is controlled using a plurality of pacing parameters at 725. Such pacing parameters include, but are not limited to, one or more of pulse amplitude, pulse width, pacing rate, pacing duration, and pacing duty cycle. Following the delivery of the electrical stimulation pulses, the preconditioned cells are selected according to one or more predetermined criteria at 740. Selection criteria include but are not limited to expression of one or more cell surface markers, apoptotic markers, cell size, cell shape, cell density, genetic modification, or any combination thereof. The selected, preconditioned cells are prepared for implantation into the living tissue at 750, such as by filtering and/or rinsing. In one embodiment, the selected, preconditioned cells are resuspended in defined media, mixed with an injectable biomaterial, genetically modified, or mixed with another molecule, for instance, a drug or other bioactive molecule such as an antibody. The prepared cells are implanted into the living tissue at 760. In one embodiment, the prepared cells are implanted into a heart. In another embodiment, the prepared cells are implanted into the spine or pancreas. Examples of implantation of cells into living tissue are discussed in U.S. patent application Ser. No. 10/722,115.

In various embodiments, the control of the delivery of the electrical stimulation pulses at 725 includes programming one or more electrical stimulation parameter values. In various embodiments, one or more of the electrical stimulation parameters are programmed to values determined based on the objective of the in vitro conditioning, the type of the cells being conditioned, and/or the target organ into which the cells are to be implanted. In various embodiments, one or more of the electrical stimulation parameters are programmed to values suitable for altering gene expression in the cells, values suitable for enhancing survival and function of the cells after being implanted into living tissue, and/or values suitable for enhancing one or more of proliferation, differentiation, alignment, conductivity, contractility, and calcium (Ca²⁺) handling of the cells. In one embodiment, one or more of the electrical stimulation parameters are programmed to values selected to subject the cells to simulated normal cardiac electrical conditions. In another embodiment, one or more of the electrical stimulation parameters are programmed to values selected to subject the cells to simulated pathological cardiac electrical conditions, such as simulated post-MI cardiac electrical conditions and simulated cardiac electrical conditions associated with heart failure. In yet another embodiment, one or more of the electrical stimulation parameters are programmed to values selected to subject the cells to simulated normal neural electrical conditions. In yet another embodiment, one or more of the electrical stimulation parameters are programmed to values selected to promote desired cell behavior, such as survival, insulin secretion, or pancreatic beta cell differentiation. In one embodiment, one or more of the electrical stimulation parameters are programmed to values selected to enhance cardioinduction, neuroinduction, or transdifferentiation, of the cells. In one embodiment, the cells subjected to electrical stimulation parameters are electrically responsive while in other embodiments the cells subjected to electrical stimulation are not electrically responsive prior to electrical stimulation but become electrically responsive after electrical stimulation. In various embodiments, the benefits of the preconditioning can be independent of the physiological function of the cells and can be independent of the endogenous electrical environment that the cells are subjected to after transplantation. For example, the in vitro conditioning of the cells can promote a stress response that would enable the cell to survive better after being transplanted.

In one embodiment, in addition to delivering the electrical stimulation pulses at 730, mechanical conditioning is applied using a device such as mechanical stimulator 654. The mechanical conditioning is applied to the cells before, during, or after the delivery of the electrical stimulation pulses. In another embodiment, in addition to delivering the electrical stimulation pulses at 730, biological treatment is administrated to the cells using a device such as biological treatment administration module 656. The biological treatment is administrated before, during, or after the delivery of the electrical stimulation pulses. In yet another embodiment, in addition to delivering the electrical stimulation pulses at 730, both mechanical conditioning and biological treatment are applied to the cells.

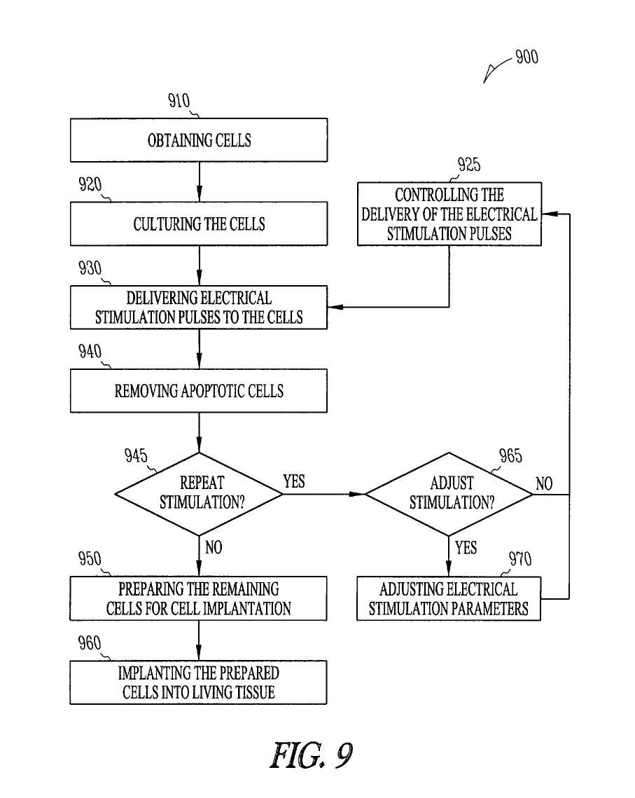

FIGS. 8-10 are each a flow chart illustrating a method being a specific embodiment of method 700. Unless specifically discussed otherwise, the discussion of method 700 above applies to each of methods 800, 900, and 1000, which are discussed below with emphasis in their differences from method 700.

FIG. 8 is a flow chart illustrating an embodiment of method 800, which is a specific embodiment of method 700. In one embodiment, in vitro cell conditioning system 100, 200, 300, or 600 is used in the performance of method 800, including steps 820, 825, and 830.

Cells are obtained at 810 for administration into living tissue. The obtained cells are cultured in a physiologic culturing medium at 820. Electrical stimulation pulses are delivered to the cells in the physiologic culturing medium at 830. The delivery of the electrical stimulation pulses are controlled using a plurality of electrical stimulation parameters at 825. Following the delivery of the electrical stimulation pulses, apoptotic cells may be separated, e.g., removed, from the conditioned cells at 840. For instance, apoptotic cells may be identified by anti-annexin V antibodies or dyes that stain apoptotic cells due to lack of membrane integrity, and then separated, for instance, by fluorescence activated cell sorting (FACS), magnetic activated cell sorting (MACS), immunoprecipitation, cell size, cell shape, cell density, mitotic state, or any combination thereof. In another embodiment, cells with a desirable phenotype are positively selected by using antibodies specific for cell surface markers associated with that phenotype. In another embodiment, undesirable cells are selected against using antibodies specific for cell surface markers associated with the undesirable cells. The remaining cells are prepared for implantation into the living tissue at 850. The prepared cells are implanted into the living tissue at 860.

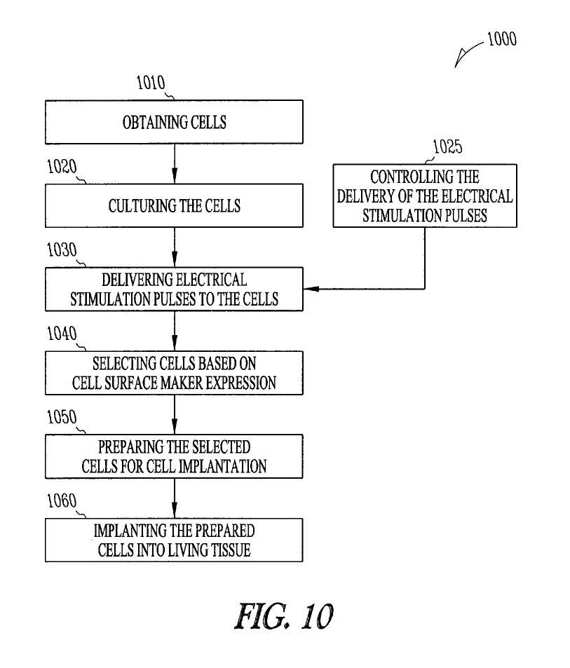

FIG. 9 is a flow chart illustrating an embodiment of method 900, which is a specific embodiment of method 700. In one embodiment, in vitro cell conditioning system 100, 200, 300, or 600 is used in the performance of method 900, including steps 920, 925, and 930.

Cells are obtained at 910 for administration into living tissue. The obtained cells are cultured in a physiologic culturing medium at 920. Electrical stimulation pulses are delivered to the cells in the physiologic culturing medium at 930. The delivery of the electrical stimulation pulses are controlled using a plurality of electrical stimulation parameters at 925. Following the delivery of the electrical stimulation pulses, apoptotic cells or other undesirable cells may be separated from the conditioned cells at 940. If the stimulation is not to be repeated at 945, the remaining cells are prepared for implantation into the living tissue at 950. The prepared cells are implanted into the living tissue at 960.

To enhance for desirable conditioned cells for implantation, steps 930 and 940 are repeated. In one embodiment, if a predetermined number of repetitions have been performed, the stimulation is not to be repeated at 945. Otherwise, the stimulation is to be repeated at 945. In one embodiment, steps 930 and 940 are repeated for two to four times. The stimulation may be repeated using different electrical stimulation parameter values. In one embodiment, the stimulation is repeated using electrical stimulation parameter values that provide for increased stimulation intensity for each repetition. If one or more the stimulation parameters are to be adjusted at 965, the one or more the stimulation parameters are adjusted to each have a new value at 970 for use in controlling the delivery of the electrical stimulation pulses at 925. If none of the stimulation parameters is to be adjusted at 965, the delivery of the electrical stimulation pulses is controlled using the parameters with unchanged values at 925.

FIG. 10 is a flow chart illustrating an embodiment of method 1000, which is a specific embodiment of method 700. In one embodiment, in vitro cell conditioning system 100, 200, 300, or 600 is used in the performance of method 1000, including steps 1020, 1025, and 1030.

Cells are obtained at 1010 for administration into living tissue. The obtained cells are cultured in a physiologic culturing medium at 1020. Electrical stimulation pulses are delivered to the cells in the physiologic culturing medium at 1030. The delivery of the electrical stimulation pulses are controlled using a plurality of electrical stimulation parameters at 1025. Following the delivery of the electrical stimulation pulses, the conditioned cells are selected based on one or more cell surface markers at 1040. In one embodiment, the conditioned cells are selected based on one or more neural cell specific surface markers. In another embodiment, the conditioned cells are selected for one or more cardiac cell specific surface markers. In a specific embodiment, the conditioned cells are selected for Cx-43, HCN or beta-adrenergic receptor expression. The selected cells are prepared for implantation into the living tissue at 1050. The prepared cells are implanted into the living tissue at 1060.

It is to be understood that the above detailed description is intended to be illustrative, and not restrictive. Other embodiments will be apparent to those of skill in the art upon reading and understanding the above description. The scope of the invention should, therefore, be determined with reference to the appended claims, along with the full scope of equivalents to which such claims are entitled.

What is claimed is:

1. A method for in vitro conditioning of mammalian cells prior to cell therapy, the method comprising:
delivering electrical stimulation pulses to the mammalian cells in a culture medium;
controlling the delivery of the electrical stimulation pulses to the mammalian cells using a plurality of electrical stimulation parameters, yielding conditioned mammalian cells; and
selecting the conditioned mammalian cells according to one or more predetermined criteria following the delivery of the electrical stimulation pulses.

2. The method of claim 1, wherein delivering the electrical stimulation pulses comprises delivering cardiac pacing pulses.

3. The method of claim 1, wherein selecting the conditioned mammalian cells comprises selecting nonapoptotic cells.

4. The method of claim 1, further comprising repeating the delivering the electrical stimulation pulses to the mammalian cells, the controlling the delivery of the electrical stimulation pulses, and the selecting the conditioned mammalian cells.

5. The method of claim 4, further comprising adjusting one or more values of the electrical stimulation parameters before the repeating the delivering the electrical stimulation pulses to the mammalian cells.

6. The method of claim 1, wherein selecting the conditioned mammalian cells comprises selecting conditioned mammalian cells having one or more predetermined cell surface markers.

7. The method of claim 6, wherein the one or more predetermined cell surface markers comprise a neural cell specific surface marker.

8. The method of claim 6, wherein the one or more predetermined cell surface markers comprise a cardiac cell specific surface marker.

9. The method of claim 1, further comprising programming the electrical stimulation parameters to values determined based on a type of the mammalian cells.

10. The method of claim 1, further comprising programming the electrical stimulation parameters to values determined based on a target organ into which the mammalian cells are to be implanted.

11. The method of claim 1, further comprising programming the electrical stimulation parameters to values suitable for altering gene expression in the mammalian cells.

12. The method of claim 1, further comprising programming the electrical stimulation parameters to values suitable for enhancing survival and function of the mammalian cells after being implanted into living tissue.

13. The method of claim 1, further comprising programming the electrical stimulation parameters to values suitable for enhancing one or more of proliferation, differentiation, alignment, conductivity, contractility, and calcium (Ca²⁺) handling of the mammalian cells.

14. The method of claim 1, further comprising programming the electrical stimulation parameters to values selected to subject the mammalian cells to simulated normal cardiac electrical conditions.

15. The method of claim 1, further comprising programming the electrical stimulation parameters to values selected to subject the mammalian cells to simulated pathological cardiac electrical conditions.

16. The method of claim 15, further comprising programming the electrical stimulation parameters to values selected to subject the mammalian cells to simulated cardiac electrical conditions after myocardial infarction.

17. The method of claim 15, further comprising programming the electrical stimulation parameters to values selected to subject the mammalian cells to simulated cardiac electrical conditions associated with heart failure.

18. A system for in vitro conditioning of mammalian cells prior to administration of the mammalian cells into living tissue, the system comprising:
a culturing container adapted to contain the mammalian cells and a culturing medium;
first and second electrodes placed in the culturing container, the first and second electrodes configured for delivering electrical stimulation pulses to the mammalian cells and including two conductive sheets placed substantially parallel to each other and on substantially horizontal planes during operation of the system; and
an electrical stimulator coupled to the first and second electrodes, the electrical stimulator adapted to deliver the electrical stimulation pulses through the first and second electrodes.

19. The system of claim 18, furthering comprising a lid configured to cover the culturing container, and wherein the first electrode is affixed to the lid.

20. The system of claim 18, wherein the mammalian cells are placed on a surface on a substantially horizontal plane, and the first and second electrodes are arranged to allow substantially uniform distribution of electrical energy associated with the electrical stimulation pulses over the substantially horizontal plane on which the mammalian cells are placed.

21. The system of claim 20, wherein the mammalian cells are placed on a surface of one of the conductive sheets.

22. The system of claim 20, wherein the two conductive sheets are separated by approximately 0.1 to 10 millimeters.

23. The system of claim 18, wherein the electrical stimulator comprises:
a pulse output circuit to deliver the electrical stimulation pulses;
an electrical stimulation controller coupled to the pulse output circuit, the electrical stimulation controller adapted to control the delivery of the electrical stimulation pulses using a plurality of electrical stimulation parameters; and
a user interface coupled to the electrical stimulation controller, the user interface including a user input device adapted to receive one or more values of the plurality of electrical stimulation parameters.

24. The system of claim 23, wherein the electrical stimulator comprises a cardiac pacemaker, the pulse output circuit comprises a pacing output circuit to deliver cardiac pacing pulses, the electrical stimulation controller comprises a pacing controller adapted to control the delivery of the cardiac pacing pulses using a plurality of pacing parameters, and the user input is adapted to receive one or more values of the plurality of pacing parameters.

25. The system of claim 24, further comprising a memory circuit coupled to the pacing controller, the memory circuit adapted to store predetermined values of the plurality of pacing parameters, and wherein the user input device is further adapted to receive selection of one or more of the predetermined values of plurality of pacing parameters stored in the memory circuit.

26. The system of claim 23, further comprising a mechanical stimulator adapted to create a mechanical stress upon the mammalian cells.

27. The system of claim 23, further comprising a biological treatment administration module adapted to introduce an exogenous agent to the culturing medium.

28. The system of claim 23, further comprising:
one or more of a mechanical stimulator and a biological treatment administration module coupled to the culturing module, the mechanical stimulator adapted to create a mechanical stress upon the mammalian cells, the biological treatment administration module adapted to introduce an exogenous agent to the culturing medium; and
a system controller coupled to the electrical stimulator and the one or more of the mechanical stimulator and the biological treatment administration module, the system controller adapted to coordinate the operation of the electrical stimulator and the one or more of the mechanical stimulator and the biological treatment administration module.
